(19) United States
(12) Patent Application Publication
(10) Pub. No.: US 2023/0240825 A1
(43) Pub. Date: Aug. 3, 2023

(54) STRIP-LIKE MEDICAL DEVICE FOR NARROWING A BODY CHANNEL

(71) Applicant: A.M.I. Agency for Medical Innovations GmbH, Feldkirch (AT)

(72) Inventors: Martin HOHLRIEDER, Götzis (AT); Martin ERHARD, Silbertal (AT)

(73) Assignee: A.M.I. Agency for Medical Innovations GmbH, Feldkirch (AT)

(21) Appl. No.: 18/012,735

(22) PCT Filed: Jun. 30, 2021
(86) PCT No.: PCT/EP2021/067988

§ 371 Date: Dec. 23, 2022

(30) Foreign Application Priority Data
Jul. 6, 2020 (AT) A150/2020

(51) Int. Cl. A61F 2/00

(52) U.S. Cl. CPC A61F 2/0036; A61F 2250/0019; A61F 2220/0033; A61F 2250/001; A61F 2230/0006

(57) ABSTRACT

A band-shaped medical device (10) for narrowing a body channel (30) of a patient, the device (10) being placeable around the body tissue surrounding the body channel (30), and the device including: a first section (1) composed of a first silicone composition (1a), and a second section (2) composed of a second silicone composition (2a) having a lower Shore A hardness compared to the first silicone composition (1a), and a connecting section (4) which integrally bonds the first section to the second section. The first section and the second section having a first locking part (11) and a second locking part (12), respectively, by which the device (10) is closable to form a ring which encircles a passage opening (16) for the body tissue surrounding the body channel (30), the connecting section (4) consisting of a mixture of the first silicone composition (1a) and the second silicone composition (2a).

### INCORPORATION BY REFERENCE

The following documents are incorporated herein by reference as if fully set forth: International Application No. PCT/EP2021/067988, filed Jun. 30, 2021; and Austrial Patent Application No. A 150/2020, filed Jul. 6, 2020.

### TECHNICAL FIELD

The invention relates to a band-shaped medical device for narrowing a body channel of a patient, the device being placeable around the body tissue surrounding the body channel, and the device comprising: a first section composed of a first silicone composition, and a second section composed of a second silicone composition having a lower Shore A hardness compared to the first silicone composition, and a connecting section which integrally bonds the first section to the second section, the first section and the second section having a first locking part and a second locking part, respectively, by means of which the device is closable to form a ring which encircles a passage opening for the body tissue surrounding the body channel.

### BACKGROUND

Band-shaped medical devices of the type mentioned are used in the human body, for example for assisting weakened natural muscles. An example of use of such devices is, for example, band-shaped devices closable to form a ring for narrowing and/or blocking a hollow organ, for example the rectum in the region of the anus.

Depending on the intended use, the different silicone compositions of which the first section and the second section of the medical device mentioned at the start consist have different mechanical properties. For example, in the case of use of the medical device to assist or to replace weakened natural muscles in the region of the anus, it is necessary, firstly, to ensure reliable functioning for treatment of fecal incontinence and, secondly, to provide a comfortable wearing feeling for the patient. This area of use requires that, for example, a relatively high elasticity of the device and, at the same time, secure locking be provided.

In the medical field, it is known to adhesively bond a first section of a medical device composed of a cured first silicone composition to a second section composed of a cured second silicone composition having a lower Shore A hardness compared to the first silicone composition. This means that, in these cases, the connecting section is formed by a thin layer of a suitable adhesive which integrally bonds the first section to the second section.

It has been found that the reliability of the adhesive bond between the two sections is often only ensured when low forces occur and that the adhesive bond is destroyed at least regionally when larger forces occur. Especially in the case of the above-illustrated use in the region of the anus, the forces that occur mean that such an adhesive bond is only usable with great care. For example, the sections to be bonded using adhesive must be provided with large overlapping regions running in the longitudinal direction of the band, as a result of which the device is stiffened with impairment of functioning and wearing comfort.

### SUMMARY

It is an object of the invention to provide an advantageous device of the type mentioned at the start that can be used reliably and is comfortable to wear for the patient.

In the case of the device according to the invention, the connecting section consists of a mixture of the first silicone composition and the second silicone composition. The connecting section which integrally bonds the first section and the second section of the device together therefore solely comprises a mixture of the first silicone composition and the second silicone composition. This means that additional adhesives or other joining means between the first section and the second section are dispensed with.

It has been found that, surprisingly, different silicone compositions which touch one another in the liquid state, for example in a mold, form upon curing a connecting section consisting of both silicone compositions. The molecules of the different silicone compositions directly crosslink and/or link with one another at the molecular level in the connecting section. As a result, the device can be exposed to large forces without tearing in the region of the connecting section.

The distribution of the portions of the first silicone composition and the second silicone composition in the connecting section is not necessarily homogeneous. In particular, the concentration of a silicone composition can increase toward the section consisting solely of said silicone composition that is connected to the connecting section, and the concentration of the other silicone composition can accordingly decrease. The connecting section could therefore also be referred to as a transition section.

By providing a first section composed of a first silicone composition and a second section composed of a second silicone composition, it is possible to provide different material properties for the particular purpose or the desired function of the device. The connecting section consisting of a mixture of the first silicone composition and the second silicone composition allows a simple design of the medical device without an accumulation of material in the region of the connecting section that bothers the patient, since, when designing a band for example, it is possible to dispense with an overlap of the sections based on the longitudinal direction of the band.

The Shore A hardness of silicone compositions is measured in the cured state, especially completely cured state, of the silicone compositions. Said state could also be referred to as the end hardness of the particular silicone composition. Shore A hardness can, for example, be determined in accordance with the standard DIN ISO 7619-1:2012-02.

Preferably, the first silicone composition has a Shore A hardness in a range from 30 to 90, preferably in a range from 40 to 80, particularly preferably in a range from 60 to 75.

The second silicone composition has advantageously a Shore A hardness lower at least by the value of 15, particularly preferably by the value of 30, compared to the first silicone composition.

The Shore A hardness of the second silicone composition is advantageously in a range from 2 to 15, preferably in a range from 3 to 10, particularly preferably in a range from 4 to 7.

In one possible embodiment, the device comprises a third section composed of a third silicone composition and an additional connecting section, the additional connecting section integrally bonding the third section to the second section and the additional connecting section consisting of a mixture of the third silicone composition and the second silicone composition. The third silicone composition can at best have a greater Shore A hardness than the second section; in particular, the third silicone composition can be identical to the first silicone composition.

Preferably, the third silicone composition has a Shore A hardness in a range from 30 to 70, particularly preferably in a range from 40 to 60.

Possible areas of use of the band-shaped medical device placeable around the body tissue surrounding the body channel are, for example, artificial sphincters for narrowing the rectum or the ureter. As a further possible area of use, the device could be placed around the esophagus in the region of the cardia as an artificial lower esophageal sphincter. The device closed to form a ring encircles the passage opening for the body tissue, for example weakened natural muscles, surrounding the body channel.

The first and second section and the optionally provided third section are advantageously arranged one after the other, viewed in a direction of longitudinal extent of the band-shaped device.

It is possible that the first locking part has an insertion opening. The first locking part having an insertion opening could also be referred to as an eyelet.

Furthermore, it is possible that the second locking part is insertable into the insertion opening and is latchable with respect to the first locking part in the closed state. As a result of being composed of the harder first silicone composition, the first locking part arranged on the first section allows reliable closure of the band-shaped device closable to form a ring.

In a possible embodiment, the second locking part can have at least two latching lugs which are latchable with the first locking part as desired to adjust the size of the passage opening. This allows adaptation of the size of the passage opening to the individual patient or to the dimensions of the body channel to be encircled.

Advantageously, the device is in the form of a single piece. This allows a simple design of the device.

The silicone compositions are advantageously so-called medical-grade silicone, which is body-compatible. The silicone compositions can be in the form of one-component or two-component silicone.

The invention further relates to a method for producing a medical device, in which the first silicone composition is filled in a liquid state into an accommodation space of a mold via a first fill opening leading to the region of the first section to be formed and the second silicone composition is filled in a liquid state into the accommodation space of the mold via a second fill opening leading to the region of the second section to be formed, the first silicone composition and the second silicone composition touching one another in the accommodation space in the liquid state and mixing with one another over the region of the connecting section to be formed. The medical device is formed by curing, especially jointly, of the first silicone composition in the first section of the device and of the second silicone composition in the second section of the device and of the mixture of the first silicone composition and the second silicone composition in the connecting section.

If the medical device additionally comprises a further section, for example the above-mentioned third section composed of a third silicone composition, the third silicone composition should accordingly be filled in a liquid state into the accommodation space of the mold via a third opening leading to the region of the third section to be formed. The second silicone composition and the third silicone composition touch one another in the accommodation space in the liquid state and mix with one another over the region of the additional connecting section to be formed. The medical device comprising three sections should then accordingly additionally be formed by curing of the third silicone composition in the third section and of the mixture of the second silicone composition and the third silicone composition in the additional connecting section.

The silicone compositions can be cured in an oven.

Optionally, it is conceivable that a slidably mounted separating slide is arranged in the region of whichever connecting section is to be formed. The separating slide divides the accommodation space into partial accommodation spaces and can be removed from the accommodation space of the mold after filling has been carried out with the silicone compositions, said silicone compositions touching one another and mixing with one another in the connecting section after the separating slide has been removed.

The length of the connecting section and/or the additional connecting section of the device is advantageously 1 mm, preferably at least 3 mm, measured in the longitudinal direction of the band-shaped device. Preferably, the length of the connecting section and/or the additional connecting section extends over less than 1.5 cm, particularly preferably less than 1 cm, in the longitudinal direction of the band-shaped device.

### BRIEF DESCRIPTION OF THE DRAWINGS

Further features and details of the invention will be explained on the basis of the exemplary embodiment of a medical device according to the invention that is shown in the figures, and the examples of method steps according to the invention for producing the device that are shown in the figures. In the figures:

FIG. 1 shows a section through a mold for producing a medical device according to the invention, wherein a first silicone composition has been filled into the mold;

FIG. 2 shows an illustration analogous to FIG. 1, in which a portion of a second silicone composition has already been filled into the mold;

FIG. 3 shows an illustration analogous to FIG. 1, in which complete filling has been carried out with the first and second silicone composition;

FIG. 4 shows the cured and demolded medical device according to FIG. 3;

FIGS. 5 and 6 show an alternative embodiment of a mold for producing a device according to the invention comprising a separating slide;

FIG. 7 shows an isometric illustration of a medical device according to the invention for forming an artificial sphincter;

FIG. 8 shows a frontal view of the device according to FIG. 7;

FIG. 9 shows a plan view of the device according to FIG. 7;

FIG. 10 shows the device according to FIG. 7 in a closed state;

FIG. 11 shows the device according to FIG. 10 in the implanted state;

FIG. 12 shows a schematic illustration of the device implanted in the human body;

FIG. 13 shows a mold for producing the medical device according to FIGS. 7-12, and

FIG. 14 shows section A-A of FIG. 13.

### DETAILED DESCRIPTION

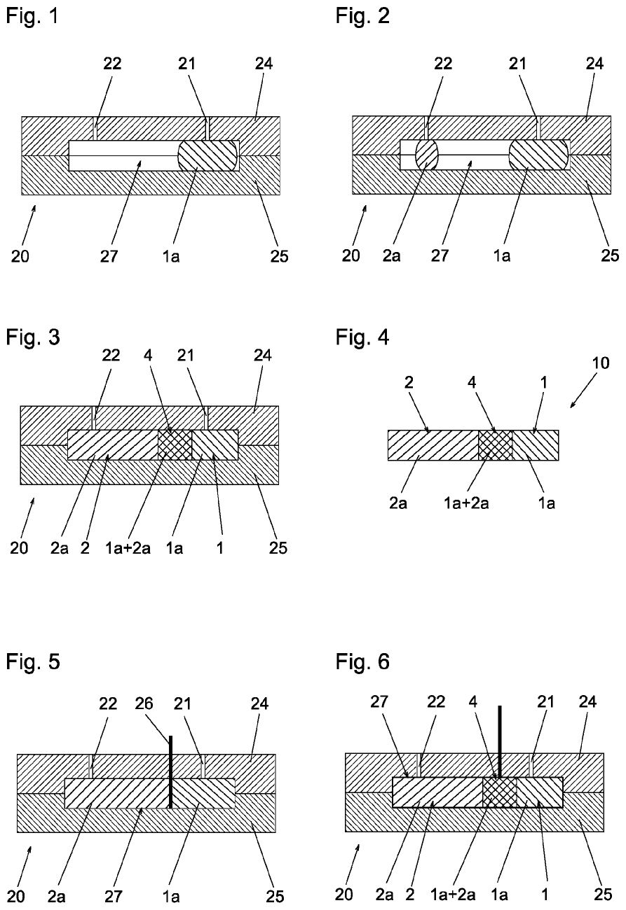

The following explanations will first deal with a method according to the invention for producing a medical device according to the invention, which medical device is depicted with shading and in greatly simplified form in FIG. 4. The different silicone compositions are depicted with different shading in FIGS. 1 to 4. For the sake of clarity, the illustration of metering pumps for carrying out filling with silicone compositions, etc., was dispensed with in FIGS. 1 to 3. Such devices are well known.

FIG. 1 depicts a mold 20 for producing a medical device 10 that comprises a bottom mold half 25 and a top mold half 24 removable from the bottom mold half 25. Arranged between the mold halves 24, 25 is an accommodation space 27.

In the exemplary embodiment, a first fill opening 21 and a second fill opening 22, which lead into the accommodation space 27, are provided in the top mold half 24. The first fill opening 21 is arranged in a region of a first section 1 of the medical device 10 that is to be formed and the second fill opening 22 is arranged in a region of a second section 2 of the medical device 10 that is to be formed.

In a first step, a silicone composition 1a is filled in a liquid state into the accommodation space 27 of the mold 20 via the first fill opening 21, cf. FIG. 1. The volume of the first silicone composition 1a filled into the accommodation space 27 is predeterminable by means of an appropriate metering pump. In FIG. 1, it is evident that the first silicone composition 1a only occupies a portion of the accommodation space 27.

Furthermore, a liquid second silicone composition 2a is now filled into the accommodation space 27 via the second fill opening 22, cf. FIG. 2. The second silicone composition 2a differs from the first silicone composition 1a with respect to the mechanical properties which the second silicone composition 2a acquires in the cured or hardened state. In particular, the second silicone composition 2a has a lower Shore A hardness than the first silicone composition 1a.

While filling is being carried out with the second silicone composition 2a, the fill opening 21 is closed or the metering pump for supplying the first silicone composition 1a is blocked. This prevents the first silicone composition 1a from being displaced from the accommodation space 27 via the first fill opening 21 when introducing the second silicone composition 2a.

FIG. 3 depicts a state in which the second silicone composition 2a has been completely filled into the accommodation space 27 and completely occupies, together with the first silicone composition 1a, the accommodation space 27.

The first silicone composition 1a and the second silicone composition 2a touch one another in the accommodation space 27 in the liquid state and mix with one another in a region of a connecting section 4 to be formed, cf. FIG. 3. In the figures, the mixture of the first silicone composition 1a with the second silicone composition 2a is identified by 1a+2a.

Air present in the accommodation space 27 before filling is carried out can escape, while filling is being carried out with the silicone compositions 1a, 2a, via the separation faces between the top mold half 24 and the bottom mold half 25, which separation faces are not described in further detail.

The medical device 10 is formed by curing, jointly, of the first silicone composition 1a in the first section 1 of the device 10 and of the second silicone composition 2a in the second section 2 of the device 10 and of the mixture 1a+2a of the first silicone composition 1a and the second silicone composition 2a in the connecting section 4. Complete curing can be effected by so-called annealing at elevated temperature, for example in a vacuum oven, as is known per se.

In the connecting section 4, crosslinking and/or linking of the molecular chains of the first silicone composition 1a and the second silicone composition 2a occurs during curing of the silicone compositions 1a, 2a. This means that a connection between the first silicone composition 1a and the second silicone composition 2a is formed in the connecting section 4 at the molecular level. The mechanism of curing, i.e., whether, for example, crosslinking occurs or linking occurs and/or both occur, depends on the properties of the respective silicone composition. The first silicone composition 1a and the second silicone composition 2a are generally not homogeneously distributed in the connecting section 4. In practice, the concentration of the individual components in relation to the sections 1, 2 containing the respective component in pure form that are connected to the connecting section 4 will generally be higher. In particular, the hardness of the connecting section 4 can change continuously over the extent thereof from the hardness of the first section 1 to the hardness of the second section 2, though the change in hardness does not necessarily have to proceed linearly. The crosslinking and/or linking of the first silicone composition 1a with the second silicone composition 2a at the molecular level achieves a permanent and highly stressable integral bond between the first section 1 and the second section 2 of the medical device 10.

In the exemplary embodiment, the first silicone composition 1a in the first section 1 of the device 10 is in pure form. This means that no other material compositions or foreign bodies are present in the first section 1 of the medical device 10. In the second section 2 too, only the second silicone composition 2a is present. The connecting section is solely composed of a mixture of the first silicone composition 1a and the second silicone composition 2a. This means that further joining means, such as adhesives for example, or other additives are dispensed with in the connecting section 4.

In the completely cured state of the device 10, cf. FIG. 4, the first and second silicone compositions 1a, 2a have their respective end hardnesses. The choice of silicone compositions 1a, 2a means that the first section 1 is harder than the second section 2. This means that the device 10 has regions of differing mechanical properties that are configured depending on the requirements of the device 10.

Preferably, the first silicone composition has a Shore A hardness in a range from 30 to 90, and the second silicone composition has a Shore A hardness in a range from 2 to 15. These ranges of values for Shore A hardness are advantageous for a broad spectrum of uses in the field of implants. Depending on what is required, it is, however, also possible to use silicone compositions having values deviating therefrom.

After curing, the medical device 10 is in single-piece form.

FIGS. 5 and 6 show an alternative to the method depicted in FIGS. 1 to 3. The structural design of the mold 20 substantially corresponds to that of the first exemplary embodiment, and so the explanations in relation to this alternative will mainly point out the differences compared to the exemplary embodiment depicted in FIGS. 1 to 3. Apart from the differences cited in what follows, the explanations in relation to the first exemplary embodiment of the method for producing the medical device 10 also apply to the second exemplary embodiment.

In the second exemplary embodiment, the mold 20 comprises a separating slide 26 which can divide the accommodation space 27 in a filled state of the mold 20 into two partial accommodation spaces, cf. FIG. 5. FIG. 5 depicts the state in which the first silicone composition 1a and the second silicone composition 2a have already been filled into the accommodation space 27. To this end, each of the components has been filled into one of the two partial accommodation spaces. The separating slide 26 separates the two silicone compositions 1a, 2a in the filled state from one another.

In the exemplary embodiment depicted, the slidably mounted separating slide 26 can be completely moved out of the region of the accommodation space 27, cf. FIG. 6. After the separating slide 26 has been slid out of the region of the accommodation space 27, the connecting section 4, in which the first silicone composition 1a and the second silicone composition 2a touch one another and mix with one another, is formed. Curing of the silicone compositions 1a, 2a is effected analogously to the above-cited example according to FIGS. 1 to 3.

The medical device 10 produced by the alternative method according to FIGS. 5 and 6 is substantially identical to the device of the variant explained above that is depicted in FIG. 4. However, the provision of a separating slide 26 means that the position of the connecting section 4 of the medical device 10 can be defined more precisely without any need to supply a certain volume of one of the two silicone compositions 1a, 2a into the accommodation space 27, as realized by means of the metering pumps in the first example.

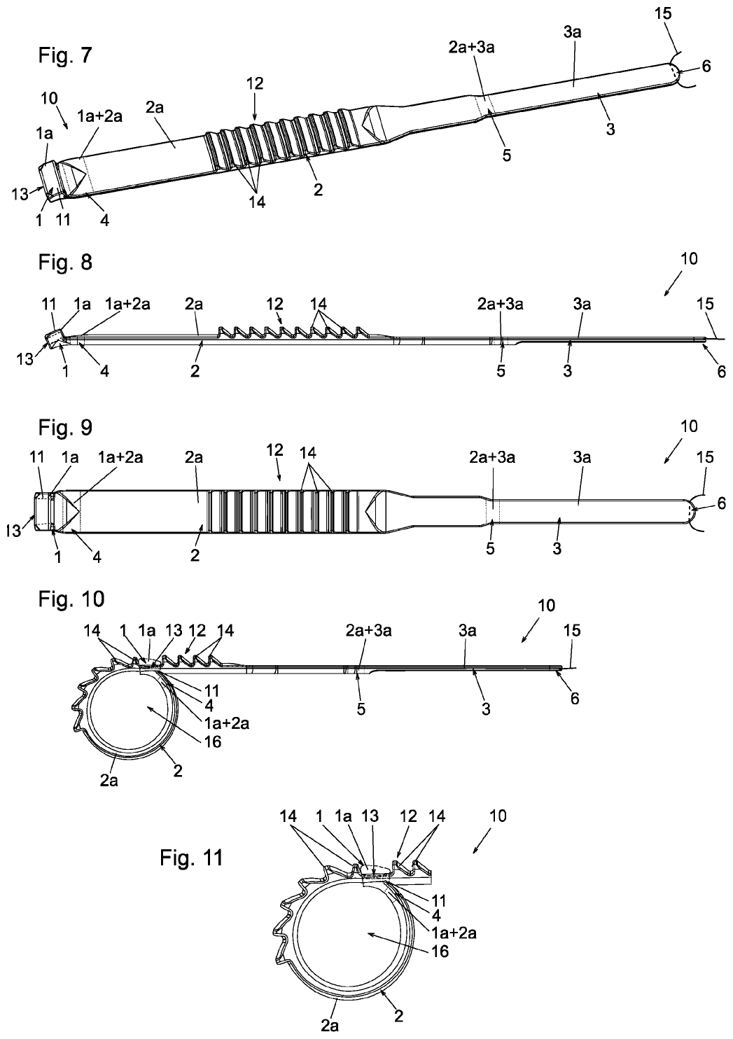

In FIGS. 7 to 12, there is now depicted a specific example of a medical device which can be used for narrowing a body channel. The structural design of the medical device with respect to the first and second section and the respective silicone compositions and to the connecting section fundamentally corresponds to the exemplary embodiment shown in FIG. 4. In what follows, differences or additional features in comparison with the example shown in FIG. 4 will therefore be mainly examined. Especially in relation to producing the medical device, reference is fundamentally made to the explanations in relation to FIGS. 1 to 3.

The medical device depicted in FIGS. 7 to 12 is intended for narrowing a body channel 30. The medical device 10 again comprises a first section 1 composed of a first silicone composition 1a and a second section 2 composed of a second silicone composition 2a having a lower Shore A hardness compared to the first silicone composition 1a. Arranged between the first section 1 and the second section 2 is a connecting section 4 which integrally bonds the first section 1 and the second section 2 together, analogously to the example according to FIG. 4.

In addition, the device 10 comprises a third section 3 composed of a third silicone composition 3a. Arranged between the second section 2 and the third section 3 is an additional connecting section 5 consisting of a mixture of the second silicone composition 2a and the third silicone composition 3a. In the figures, the mixture of the second and third silicone composition 2a, 3a is identified by 2a+3a.

In order to be able to visually distinguish the sections 1-3 and the connecting sections 4, 5 of the device 10 from one another in FIGS. 7-10, they are depicted with delimitation from one another by dotted lines.

In the exemplary embodiment according to FIGS. 7-12, the third silicone composition 3a is identical to the first silicone composition 1a. The second silicone composition 2a therefore has a lower Shore A hardness compared to the first silicone composition 1a and the third silicone composition 3a.

The second silicone composition 2a has a Shore A hardness lower at least by the value of 30 compared to the first silicone composition 1a. In the exemplary embodiment, the hardness of the first silicone composition 1a is 70 Shore A. The third silicone composition 3a accordingly also has a Shore A hardness of 70 in the exemplary embodiment.

The second silicone composition 2a advantageously has a Shore A hardness in a range from 2 to 15. In the exemplary embodiment, the hardness of the second silicone composition 2a is 5 Shore A. Silicone compositions having a Shore A hardness in the stated range from 2 to 15 Shore A are substantially more flexible in comparison with silicone compositions having a Shore A hardness from 40 to 90.

The device 10 is band-shaped and placeable around the body tissue surrounding the body channel 30. The first section 1 and the second section 2 of the device 10 have a first locking part 11 and a second locking part 12, respectively, by means of which the device 10 is closable to form a ring which encircles a passage opening 16 for the body channel 30.

The first locking part 11 has an insertion opening 13 into which the second locking part 12 is insertable. The second locking part 12 has a multiplicity of latching lugs 14 which, in the closed state of the device 10 with respect to the first locking part 11, are latchable with the first locking part 11 as desired to adjust the size of the passage opening 16, cf. FIGS. 10 and 11. Owing to the low Shore A hardness of the second silicone composition 2a, the second locking part 12 can be pulled through the insertion opening 13 by the operator as a result of elastic deformation of the latching lugs 14.

The third section 3 of the device forms a band-shaped manipulation section for passing the second locking part 12 through the insertion opening 13. In the exemplary embodiment, the cross section of the manipulation section is smaller than the cross section of the insertion opening 13. This means that the manipulation section is insertable through the insertion opening with play, which facilitates the threading of the third section 3 through the first locking part 11 during implantation.

In the exemplary embodiment, the device comprises an additional insertion section 6 in which a manipulation thread 15 is arranged. The insertion section 6 is materially integrally connected to the third section 3 of the device 10 and comprises a main body which is made of the third silicone composition 3a and into which the manipulation thread 15 has been embedded. The manipulation thread 15 can be grasped by the operator by means of an appropriate instrument and simplifies the implantation of the device 10.

Overall, the medical device according to FIGS. 7-12 is in the form of a single piece. Apart from the optional manipulation thread 15, the medical device 10 consists entirely of silicone compositions.

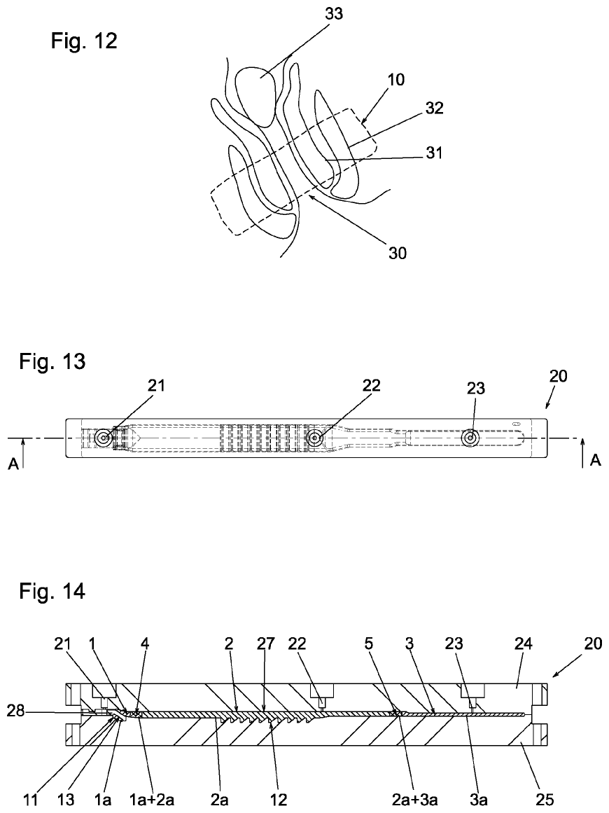

FIG. 12 schematically depicts the use of the device 10 as an artificial anal sphincter in the implanted state in order to illustrate the function of the device as an artificial sphincter. The medical device 10 is placed around the internal anal sphincter 31 and the external anal sphincter 32 and assists, in the closed state in which said device is closed to form a ring, the narrowing of the body channel 30, i.e., the rectum. Such medical devices 10 are, as explained at the start, also referred to as an anal band for treatment of fecal incontinence.

The third section 3 and the insertion section 6, and regions of the second section 2 not further required for the purpose of the device 10, can be removed by the operator after the ring has been closed. This means that the implanted device 10 is conveniently shortened to the essential length required for the function to be provided. In the implanted state, the medical device 10 used this way merely comprises the first section 1 and a portion of the original second section 2 and also the connecting section 4 in between, cf. FIG. 11.

In the implanted state, the soft second silicone composition 2a allows elastic expansion of the medical device 10 in the radial direction. This means that the passage opening 16 of the device 10 is enlargeable for defecation of stool 33. After the stool 33 has been defecated from the rectum, what occurs is elastic recovery of the device 10, which assists the closure of the rectum. In particular, the medical device 10 assists the weakened anal sphincters 31, 32.

As is evident in FIG. 11, the second section 2 of the device in the exemplary embodiment, when the device 10 has been closed to form a ring, extends over at least 75% of the circumference of the passage opening 16.

The harder silicone composition 1a ensures secure locking of the device 10 closed to form a ring when the device has been implanted. In the exemplary embodiment, the first section 1 extends over less than 20% of the circumference of the passage opening 16 when the device 10 has been closed to form a ring. A small extent of the first section 1 improves the wearing comfort of the device 10.

FIGS. 13 and 14 depict the mold 20 by means of which the medical device 10 of FIGS. 7 to 12 can be produced. With respect to the structural design, this mold 20 has numerous similarities to the example schematically depicted in FIGS. 1 to 3, and so the following discussions will mainly deal with differences in comparison with the mold 20 depicted in FIGS. 1 to 3. Apart from the differences cited in what follows, the explanations in relation to FIGS. 1 to 3 also apply to the mold 20 depicted in FIGS. 13 and 14.

The mold 20 depicted in FIGS. 13 and 14 comprises a top mold half 24 having a first fill opening 21, a second fill opening 22 and a third fill opening 23, which fill openings lead to the accommodation space 27. Furthermore, the mold 20 comprises an insert 28 which allows removal of the medical device 10 from the mold in the solidified state. Furthermore, the insert 28 serves to form the insertion opening 13 of the medical device 10, cf. FIG. 14. Inserts are fundamentally known from the field of injection-molding molds, for example for production of undercuts.

FIG. 14 depicts a state in which the first silicone composition 1a and the second silicone silicone composition 2a and the third silicone composition 3a have been filled into the accommodation space 27 in the liquid state. FIG. 14 also depicts the connecting section 4, in which the first silicone composition 1a and the second silicone composition 2a have been mixed, and the additional connecting section 5, in which the second silicone composition 2a and the third silicone composition 3a have been mixed together.

After the medical device 10 has cured, the top mold half 24 can be removed from the bottom mold half 25 and the insert 28 can be removed from the insertion opening 13 of the first locking part 11 of the device 10. Optionally, the medical device 10 is annealed after the device 10 has been removed.

In another use, the device shown in FIGS. 7-12 10 could be analogously used as an artificial esophageal sphincter in the region of the esophagus.

When using the device 10 as an anal band, as shown in FIGS. 7-12, elastic stretchability of the device is essential, and this is why the second section 2 in the closed state extends over the predominant part of the circumference of the passage opening 16. In other uses of the device, it would, however, also be conceivable and possible for a substantially rigid device to be provided, for example as reinforcement or fortification of a hollow organ. It is then conceivable and possible that the harder silicone composition extends over the predominant part of the circumference of the passage opening. For example, the first locking part could be formed from a silicone composition that is less hard than the second locking part, providing a certain elasticity which improves the function of the locking mechanism.

In other uses, it would be conceivable and possible that yet further sections of differing silicone composition are provided. This is guided by the medical requirements of the device to be implanted. Advantageously, in these variants as well, what are arranged between the further sections composed of different silicone compositions are corresponding connecting sections or additional connecting sections, each of which consists of a mixture of the silicone compositions used in the respectively adjacent sections. In addition, optionally provided locking parts can be provided on any of the sections of the device.

### KEY TO REFERENCE SIGNS

1 First section

1a First silicone composition

2 Second section

2a Second silicone composition

3 Third section

3a Third silicone composition

4 Connecting section

5 Additional connecting section

6 Insertion section

10 Device

11 First locking part

12 Second locking part

13 Insertion opening

14 Latching lug

15 Manipulation thread

16 Passage opening

20 Mold

21 First fill opening

22 Second fill opening

23 Third fill opening

24 Top mold half

25 Bottom mold half

26 Separating slide

27 Accommodation space

28 Insert

30 Body channel

31 Internal anal sphincter

32 External anal sphincter

33 Stool

1. A band-shaped medical device for narrowing a body channel of a patient, the device being placeable around body tissue surrounding the body channel, the device comprising:
a first section formed of a first silicone composition;
a second section formed of a second silicone composition having a lower Shore A hardness compared to the first silicone composition;
a connecting section which integrally bonds the first section to the second section; and
the first section and the second section having a first locking part and a second locking part, respectively, by which the device is closable to form a ring which encircles a passage opening for the body tissue surrounding the body channel; and
wherein the connecting section consists of a mixture of the first silicone composition and the second silicone composition.

2. The device as claimed in claim 1, wherein the second silicone composition has a Shore A hardness lower at least by a value of 15 compared to the first silicone composition.

3. The device as claimed in claim 1, wherein the first silicone composition has a Shore A hardness in a range from 30 to 70, and the second silicone composition has a Shore A hardness in a range from 2 to 15.

4. The device as claimed in claim 1, wherein the first locking part has an insertion opening.

5. The device as claimed in claim 4, wherein the second locking part is insertable into the insertion opening and is latchable with respect to the first locking part in a closed state.

6. The device as claimed in claim 5, wherein the second locking part has at least two latching lugs which are latachable with the first locking part as desired to adjust a size of the passage opening.

7. The device as claimed in claim 1, further comprising a third section formed of a third silicone composition and an additional connecting section which integrally bonds the third section to the second section, the additional connecting section consisting of a mixture of the second silicone composition and the third silicone composition.

8. The device as claimed in claim 7, wherein the first locking part has an insertion opening, and the third section forms a band-shaped manipulation section for passing the second locking part through the insertion opening.

9. The device as claimed in claim 7, wherein the third silicone composition has a Shore A hardness in a range from 30 to 70.

10. The device as claimed in claim 1, wherein the device is in the form of is formed as a single piece.

11. A method for producing a medical device as claimed in claim 1, the method comprising:
filling the first silicone composition in a liquid state into an accommodation space of a mold via a first fill opening leading to a region of the first section to be formed;
filling the second silicone composition in a liquid state into the accommodation space of the mold via a second fill opening leading to a region of the second section to be formed, with the first silicone composition and the second silicone composition touching one another in the accommodation space in the liquid state and mixing with one another over a region of the connecting section to be formed; and
forming the medical device by curing of the first silicone composition in the first section of the device and curing of the second silicone composition in the second section of the device and of the mixture of the first silicone composition and the second silicone composition in the connecting section.
